(19) United States
(12) Patent Application Publication
(10) Pub. No.: US 2023/0058895 A1
(43) Pub. Date: Feb. 23, 2023

(54) PHYSIOLOGICALLY ACCEPTABLE EUTECTIC MIXTURES OF CANNABIDIOL

(71) Applicants: Joseph Schwarz, Toronto (CA); Michael Weisspapir, Toronto (CA)

(72) Inventors: Joseph Schwarz, Toronto (CA); Michael Weisspapir, Toronto (CA)

(21) Appl. No.: 17/881,806

(22) Filed: Aug. 5, 2022

Related U.S. Application Data
(60) Provisional application No. 63/230,679, filed on Aug. 6, 2021.

(51) Int. Cl. A61K 31/05; A61K 47/10; A61K 47/14; A61K 47/22; A61K 47/24; A61K 47/26

(52) U.S. Cl. CPC A61K 31/05; A61K 47/10; A61K 47/14; A61K 47/22; A61K 47/24; A61K 47/26

(57) ABSTRACT

Pharmaceutical compositions comprising eutectic mixtures comprising Cannabidiol (CBD) and at least one physiologically acceptable organic compound, allowing to increase solubility of cannabinoid in pharmaceutical compositions and to improve drug loading, drug absorption and bioavailability of CBD after administration. Methods of increasing cannabidiol (CBD) solubility for use in pharmaceutical formulations with high CBD loading

### FIELD

The present invention relates to the field of increasing cannabidiol (CBD) solubility for use in pharmaceutical formulations with high CBD loading. The present invention comprises a eutectic mixture comprising cannabidiol (CBD) and at least one physiologically acceptable organic compound, allowing to increase solubility of cannabinoid in pharmaceutical compositions and to improve drug loading, drug absorption and bioavailability of CBD after administration.

### BACKGROUND

Eutectic mixture (EM) is defined as a homogeneous mixture of two or more components that melts or solidifies at a single temperature that is lower than the melting point of any of the individual constituents. One of the most important characteristics of eutectics is their capability to melt/freeze congruently without phase segregation.

There are few examples of eutectic mixtures, comprising active pharmaceutical ingredients (API). A typical example of a eutectic mixture in pharmaceutics is combination of two local anaesthetics, Lidocaine and Prilocaine bases (EMLA). Both compounds are solid at room temperature (melting points 68° C. and 37° C., respectively) while the mixture 1:1 melts at 18° C. Cream-gel based on the emulsion of the liquid EMLA combination (oil phase) successfully used as an efficient topical anaesthetic formulation with enhanced activity.

Gala et al., in “Pharmaceutical Applications of Eutectic Mixtures”; J. Develop. Drugs 2013, vol. 2 issue 3, pp. 1-2 (DOI: 10.4172/2329-6631.1000e130) describes several API based eutectic mixtures, mostly solid, (e.g., Curcumin with Nicotinamide; Ibuprofen with Thymol; Genistein with Polyethylene glycol) demonstrating improved dissolution and absorption.

Yong et al. in “Improved Solubility and In Vitro Dissolution of Ibuprofen from Poloxamer Gel Using Eutectic Mixture with Menthol”; Drug Delivery, (2003) 10:3, pp. 179-183, DOI: 10.1080/713840406 demonstrated improved solubility of Ibuprofen eutectic mixture with menthol in water solutions of poloxamer 188.

Bazzo et al. in the review “Eutectic mixtures as an approach to enhance solubility, dissolution rate and oral bioavailability of poorly water-soluble drugs” International Journal of Pharmaceutics 588 (2020) 119741 (doi.org/10.1016/j.ijpharm.2020.119741) describes numerous combinations of API with physiologically acceptable compounds (e.g., glimepiride and arginine, nimesulide and nicotinamide, curcumin and salicylic acid, etc.) with improved solubilities.

Kaplun-Frischov et al. in “Testosterone Skin Permeation Enhancement by Menthol through Formation of Eutectic with Drug and Interaction with Skin Lipids” Journal of Pharmaceutical Sciences 86(12), 1394-1399. doi:10.1021/js9701465 describes formation of eutectic mixture between menthol and testosterone with enhanced transdermal penetration. Also, Lodzki et al. in “Cannabidiol—transdermal delivery and anti-inflammatory effect in a murine model” Journal of Controlled Release 93 (2003) 377-387 (doi:10.1016/j.jconrel.2003.09.001) described use of eutectic mixture of CBD with lecithin in lecithin-ethanol ethosomes for topical applications.

Draper in the US Patent application US2007/0224261A1 described eutectic mixtures of ibuprofen with menthol, thymol and some terpenes forming a liquid EM with high level of drug loading and suitable for administration vial oral and topical route. No examples of preparation, as well as no efficacy or absorption improvement of Ibuprofen in EM were provided.

Eutectic mixtures of sugars or polyols with organic acids, inorganic salts and water [US Pat App. 2014/0341934] recommended for improved extraction of cannabinoids from plants.

CBD is a biologically active cannabinoid. Due to interaction with CD receptors of endocannabinoid system CBD can be used for treatment of inflammatory conditions, anxiety, seizures, as sleeping aid in topical formulations for local inflammation. Since the binding constant of CBD with these receptors is low, the dose required for revealing of biological activity of CBD is higher than doses of THC. For example, CBD for treatment of epilepsy should be given to human patients in doses up to 1800 mg/day or even more (up to 20 mg/kg/day). It makes incorporation of required amounts of CBD into solid dosage forms such as capsules or tablets very complicated.

CBD has relatively good solubility in lipids, but usually not high enough to provide required drug loading for absorbable solid dosage forms. Since CBD in existing oral dosage forms (e.g., Epidiolex®—10% CBD solution in sesame oil with ethyl alcohol, sweetener and flavor; multiple examples of medicinal cannabis formulations in soft gelatin capsules, compressed tablets and different edibles) demonstrates low bioavailability (BA)—just about 6% [Millar S. et al., Towards Better Delivery of Cannabidiol (CBD). Pharmaceuticals 2020, vol. 13, pp. 219]. There is an unmet need in development of the effective method for increasing CBD solubility for use in pharmaceutical formulations with high CBD loading.

### SUMMARY

Provided herein are methods and compositions for increasing CBD solubility for use in pharmaceutical formulations with high CBD loading. In particular, the present invention comprises a eutectic mixture comprising cannabidiol (CBD) and at least one physiologically acceptable organic compound, allowing to increase solubility of cannabinoid in pharmaceutical compositions and to improve drug loading, drug absorption and bioavailability of CBD after administration.

### BRIEF DESCRIPTION OF FIGS

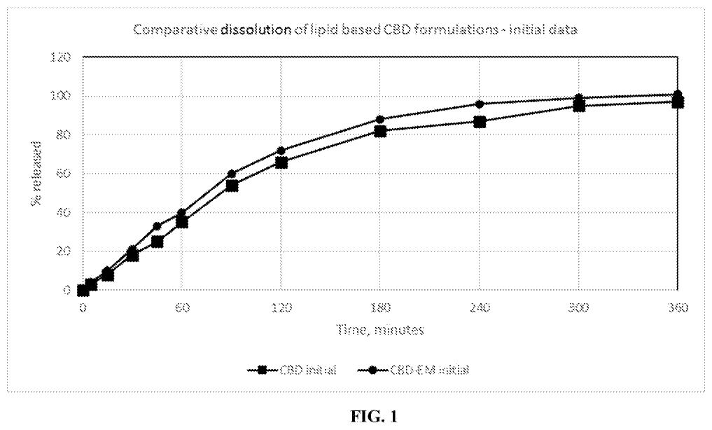

FIG. 1 is a graphical plot showing the dissolution of the CBD lipid formulation (initial data).

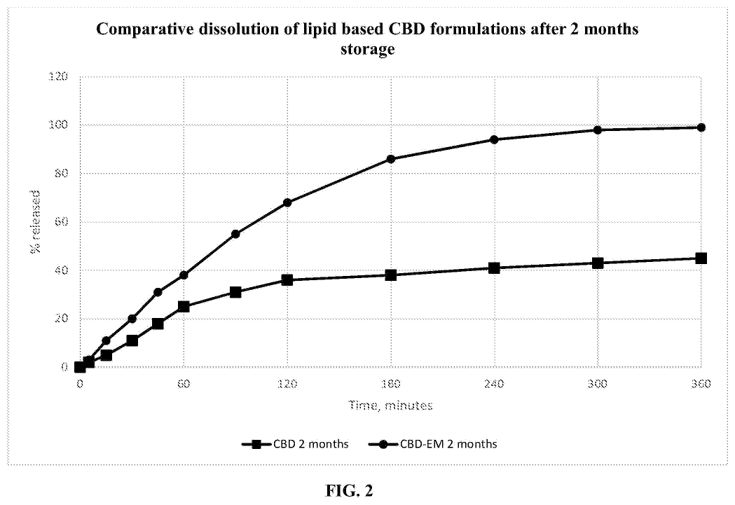

FIG. 2 is a graphical plot showing dissolution of CBD lipid formulation after 2 months of storage at ambient temperatures.

### DETAILED DESCRIPTION OF THE INVENTION

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range, Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, and each separate value is incorporated into the specification as if it were individually recited herein.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μNI, etc.).

The term “about,” as used herein, is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

It was surprisingly found that CBD (melting point 66-68° C.) forms eutectic mixtures (EMs) with different physiologically acceptable safe and non-toxic hydroxyl containing organic compounds, such as fatty alcohols, cyclic alcohols, fatty acids, aromatic alcohols and acids, phenolic compounds, glycerides, polyethoxylated fatty acids and polyethoxylated glycerides, polyethylene glycols, tocopherols and tocopherol derivatives, and other compounds. The formed EMs have melting points lower than CBD and other component of the eutectic composition.

These eutectic mixtures for CBD and organic compounds could be formed in wide range of combinations, e.g., with molar ratios between 1:10 to 10:1, respectively.

Eutectic mixtures (EM) were prepared by combining of the compound with CBD and heating to temperature, slightly higher than melting point of the lowest melting component. After cooling and equilibration, the formed EM has melting point or glass transition temperature lower than any of each of its components.

CBD containing EM demonstrated good compatibility with pharmaceutical excipients such as lipids and surfactants and increased solubility of CBD.

The detailed description set forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent or bioequivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

Also provided are embodiments wherein any embodiment above can be combined with any one or more of these embodiments, provided the combination is not mutually exclusive. Also provided herein are uses in the treatment of indications or one or more symptoms thereof as disclosed herein, and uses in the manufacture of medicaments for the treatment of indications or one or more symptoms thereof as disclosed herein, equivalent in scope to any embodiment disclosed above, or any combination thereof that is not mutually exclusive. The methods and uses may employ any of the devices disclosed herein, or any combination thereof that is not mutually exclusive, or any of the pharmaceutical formulations disclosed herein, or any combination thereof that is not mutually exclusive.

### Examples

The following examples are included to demonstrate preferred embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1: Examples of Binary EM Containing CBD

| | | Molecular | Component’s | EM | Molar ratios |
|---|---|---|---|---|---|
| | | mass, | melting | melting | COMPONENT: |
| | COMPONENT | Dalton | point, ° C. | point, ° C. | CBD |
| | CANNABIDIOL (CBD) | 314.5 | 66-68 | | |
| Ex. 1 | Hydrogenated lecithin | 748.1 | 160-270 | 46-50 | 3:1-1:2 |
| Ex. 2 | 5-Methyl-2-(isopropyl)cyclohexan-1-ol | 156.3 | 44 | <−18 | 4:1-0.5:1 |
| | (L-menthol) | | | | |
| Ex. 3 | Methyl 2-hydroxybenzoate | 152.1 | −9 | <−18 | 5:1-1:4 |
| Ex. 4 | Cetyl alcohol (C16) | 242.4 | 50 | 36-39 | 3:1-6:1 |
| Ex. 5 | Glyceryl monostearate (GMS) | 358.6 | 80 | 42-46 | 1:2-2:1 |
| Ex. 6 | Glyceryl monolinoleate (GML, Maisine CC) | 354.5 | 14-16 | −6-8 | 1:4-4:1 |
| Ex. 7 | Glyceryl monooleate (GMO) | 356.5 | 33-36 | 14-18 | 1:4-4:1 |
| Ex. 8 | Glyceryl monocaprylate (GMC) | 218.3 | 30-34 | 8-13 | 1:4-4:1 |
| Ex. 9 | Cocoa butter | 816.4 | 33-34 | 30-32 | 1:2-10:1 |
| Ex. 10 | Polysorbate-80 (PEG 20 sorbitan monooleate) | ~1310 | ~−18 | <−21 | 0.5:10-1:5 |
| Ex. 11 | Benzyl alcohol | 108.1 | −15 | <−19 | 2:1-1:5 |
| Ex. 12 | PEG-40 Hydrogenated castor oil (Kolliphor ™ RH40) | ~2100 | 18-26 | 2-9 | 1:6-1:1 |
| Ex. 13 | 12-Hydroxystearic acid (C18) | 300.5 | 74-76 | 48-52 | 0.5:1-3:1 |
| Ex. 14 | Behenyl alcohol (C22) | 326.6 | 64-70 | 52-55 | 0.5:1-1:2 |
| Ex. 15 | Vitamin E succinate PEG-1000 ester (TPGS) | 1513 | 37-41 | 12-16 | 1:5-1:1 |
| Ex. 16 | Polyethylene glycol 3350 (PEG-3350) | 3350 | 53-57 | 44-46 | 1:5-1:1 |
| Ex. 17 | d-alpha Tocopherol acetate | 472.7 | 24-26 | 22-24 | 1:1-1:2 |
| Ex. 18 | d-alpha Tocopherol | 430.7 | 1-3 | <−18 | 1:1-1:2 |
| Ex. 19 | Gelucire ™ 48/16 (PEG 32 stearate) | ~1780 | 46-48 | 22-24 | 0.5:10-1:5 |
| Ex. 20 | Mirj ™-52 (PEG 40 stearate) | ~2030 | 44-47 | 26-28 | 0.5:10-1:5 |
| Ex. 21 | Gelucire ™ 44/14 [Lauroyl (polyoxyl-32) glyceride] | ~1957 | 38-44 | 21-22 | 0.5:10-1:5 |
| Ex. 22 | Tween 60 (PS 60, PEG 20 sorbitan monostearate) | ~1311 | 45-50 | 4-8 | 0.5:10-1:5 |
| Ex. 23 | Labrasol ™ ALF (Caprylcaproyl PEG-8 glycerides) | ~617 | −3-−4 | <−18 | 1:10-1:2 |

EM of hydrogenated lecithin was prepared by solubilization of components in ethanol-dichloromethane mixture (3:1) followed by evaporation of the organic solvents at 50° C.; all other EM were obtained by dissolving of CBD in the liquid excipients or mixing components for 24 hours at temperature slightly (4-5° C.) higher than melting point (MP) of the lower melting component.

### Example 2: Examples of Ternary CBD Loaded EM

Ternary CBD loaded EM were formulated with:

Ex. 24. CBD, Cocoa butter and L-Menthol (5-Methyl-2-(isopropyl)cyclohexan-1-ol) (molar ratio 1:3:2, MP 8-11° C.);

Ex. 25. CBD, d-alpha Tocopherol and L-Menthol (molar ratio 2:1:0.5), MP<−13° C.;

Ex. 26. Cannabidiol, Menthol and Methyl 2-Hydroxybensoate (molar ratio 2:1:0.5), MP<−13° C.

Ex. 27. CBD, Maisine and L-Menthol (5-Methyl-2-isopropylcyclohexan-1-01) (molar ratio 1:1:0.2, MP<6° C.)

Ex. 28. CBD, Maisine and PEG 40 stearate (molar ratio 2:1:0.1, MP<9° C.)

Binary and ternary CBD containing EMs demonstrated increased solubility in triglycerides (e.g., CBD solubility in MCT or LCT oils at ambient temperature increased from 25-35% w/w to more than 40-50% with use of EM corresponding to examples 2-5, 7-9, 12-14, 17-19, 21-23 and 24-27 (Tables 4,5).

### Example 3: Topical Formulations

MCT based oil-in-water cream-gel type topical formulations containing CBD in EM of examples 2, 3 or 8 demonstrated much higher transdermal penetration and anti-inflammatory activity compared with similar topical creams based on MCT oil cream-gel formulation,

TABLE 1
Muscle pain suppression evaluation in volunteers for
topical lipid formulations containing CBD

| | Pain relief |
|---|---|
| Placebo | + − |
| CBD in MCT based ointment (Ex. 29) | + |
| CBD in o/w MCT based lotion (Ex. 33) | + + |
| CBD in EM cream-gel (Ex. 36) | + + + + |
| Indomethacin gel (Sumitomo) | + + |

### Example 4: Oral Formulations

Oral formulations containing CBD in eutectic mixtures showed improved absorption, compared with CBD solutions in oils (e.g., soybean oil, sesame oil or fractionated coconut oil) with identical content of CBD. Due to significantly increased solubility of CBD EMs solid dosage forms such as tablets, chewables, softgels and hard capsules with high drug loading can be effectively developed.

Since CBD molecule has extremely low solubility in water based physiological media, it is highly desirable to have the administered compound already dissolved to facilitate dissolution and absorption in the body. Incorporation of highly loaded CBD solutions, based on EMs, into appropriate delivery systems may significantly improve absorption and bioavailability.

Eutectic mixtures of CBD with non-irritant eutectic component can be effectively incorporated into rectal, ophthalmic, subcutaneous, intramuscular and intravenous formulations.

Eutectic mixtures of CBD may be successfully incorporated into self-emulsifying delivery systems (SEDS), self-nanoemulsifying delivery systems (SNEDDS), microemulsions, nanoemulsions and other colloidal delivery systems for poorly water soluble active pharmaceutical ingredients. Such delivery systems may be used for oral, rectal, transmucosal, topical, ophthalmic, parenteral administration routes.

Beside pharmaceutical applications, eutectic mixtures of CBD can be formulated as cosmetic, nutraceutical and natural health products, dietary supplements, as well as food products, beverages, concentrates and other edibles.

Incorporation of CBD into EM increases solubility and physical stability of CBD and prevents partial crystallization and precipitation of CBD during storage (see FIG. 1, FIG. 2 and Table 2). Similarly, no precipitation from highly loaded self-emulsifying formulations with CBD-EM was observed during extended storage (Table 3).

Physical Stability

Lipid formulations of CBD and CBD eutectic mixtures with different concentrations were prepared by slight heating of the oil phase with CBD and then stored at room temperatures in tightly closed vials, protected from light.

CBD is known to demonstrate oversaturation in lipid solutions, especially when the system was initially heated. Extended storage of different CBD formulations showed that formulations with EM keep CBD in solubilized stage for long time without crystallization or precipitation.

TABLE 2
Physical stability of CBD solutions

| | | 35% | | 50% |
|---|---|---|---|---|
| | | CBD solution | 50% | CBD solution |
| CBD | 35% CBD | in Soybean | CBD | in Maisine |
| conc., % | solution in | oil CBD + | solution in | CBD + |
| Time point | Soybean oil | Menthol 3:1 EM | Maisine | PS60 1:1 EM |
| Time 0 | 35% | 35% | 50% | 50% |
| 1 week at | 35% | 35% | 39% | 50% |
| RT (22° C.) | | | | |
| 1 month at | 29.1% | 35% | 34.3% | 50% |
| RT | | | | |
| 6 months | 26.4% | 35% | 33.4% | 50% |
| at RT | | | | |

Chemical Stability

CBD in EMs demonstrate excellent chemical stability for extended period of time.

TABLE 3
CBD content in 1% CBD topical cream with ternary EM (Cannabidiol, Menthol
and Methyl 2-Hydroxybensoate, molar ratio 2:1:5) after 2 years storage at ambient
conditions (+25° C., 60% RH)

| | Time = 0 | 6 months | 9 months | 12 months | 18 months | 24 months |
|---|---|---|---|---|---|---|
| CBD content | 10.24 | 10.19 | 10.22 | 10.03 | 10.09 | 10.12 |
| mg/g | | | | | | |
| % of label claim | 102.4% | 101.9% | 102.2% | 100.3% | 100.9% | 101.2% |

Increased Solubility

The EM of CBD with hydrophobic hydroxyl containing compounds demonstrate excellent solubilities in lipids.

E.g., surfactants with high HLB, such as Polysorbate 80, HLB 14-15), Polysorbate 60 (HLB 14-15), PEG 40 stearate (HLB 15-16), Labrasol™ (caprylcaproyl polyoxyl glyceride, HLB 12-14) are poorly soluble in lipids. Surprisingly it was found that eutectic mixtures of CBD and these surfactants demonstrated good compatibility with lipids, high solubilities in oils and forms clear and transparent oil solutions when CBD incorporated in EM (Tables 4 and 5).

TABLE 4
Solubility of CBD EM with polyethoxylated surfactant
(PEG 40 stearate) in Maisine CC

| | | | |
|---|---|---|---|
| CBD, mg | 0 | 300 | 350 |
| PEG 40 | 50 | 50 | 100 |
| stearate, mg | | | |
| Maisine CC | 150 | 150 | 200 |
| (GML), mg | | | |
| Solubility | N/A | 41.6% | 53.2% |
| | Phase separation, | Partial CBD | CBD |
| | most of PEG | solubilization, | completely |
| | stearate | all PEG | dissolved, clear |
| | not dissolved | stearate dissolved | solution |

TABLE 5
Solubility of CBD EM with polyethoxylated
surfactants in Soybean oil (LCT)

| | | | |
|---|---|---|---|
| CBD, mg | 0 | 300 | 350 |
| PEG 40 stearate, | | | 100 |
| mg | | | |
| Polysorbate 60 | 50 | 50 | |
| (PS-60), mg | | | |
| Soybean oil, mg | 200 | 200 | 200 |
| Solubility | N/A | 42.2% | 49.8% |
| | Cloudy mixture, | Partial CBD | All CBD |
| | tends to phase | solubilization, | dissolved, |
| | separation (LCT oil | transparent | clear |
| | incompatible | solution | solution |
| | with PS-60) | | |

### Example 5: Topical Formulations Containing CBD Eutectic Mixtures

Topical formulations with CBD eutectic mixtures were prepared by different methods and showed noticeable pain relief after application on the inflamed joints (knee, palms, fingers) while traditional marketed CBD formulations (salves, ointments, balms, oil based creams) provide only slight anti-inflammatory action.

TABLE 6
Topical CBD formulations containing CBD EM

| | Weight, mg | | | | | | | |
|---|---|---|---|---|---|---|---|---|
| Component | Ex. 29 | Ex. 30 | Ex. 31 | Ex. 32 | Ex. 33 | Ex. 34 | Ex. 35 | Ex. 36 |
| Cannabidiol crystalline | 2,000 | 2,000 | 1,000 | 2,000 | 1,000 | 1,000 | 2,000 | 5,000 |
| (99.87%) | | | | | | | | |
| Polyoxyl-35 castor oil | | | | | 5,200 | 5,000 | 5,000 | 6,500 |
| (Kolliphor EL) | | | | | | | | |
| Polysorbate 60 (Tween 60) | | | 4,200 | 5,000 | | | | |
| TPGS | | 1,000 | | | 3,100 | 4,750 | 3,500 | 5,500 |
| Cetostearyl alcohol | 6,000 | | | | | | | |
| Glyceryl monostearate | | 2,800 | | | | | | |
| Sorbitan monostearate | | 1,200 | | | | | | |
| (Span 60) | | | | | | | | |
| Ceteareth 25 | | 1,000 | | | | | | |
| Methyl salicylate | | | 2,000 | 2,500 | | | | 5,000 |
| (Wintergreen oil) | | | | | | | | |
| L-Menthol | 1,000 | 2,000 | 2,000 | | | | | 1,500 |
| rac-Camphor | | | | | | | | 1,500 |
| Peppermint oil | | | | 2,300 | 2000 | 2700 | 3,040 | 500 |
| Black pepper oil | | | | 2,000 | 2,500 | 2,500 | 2,500 | |
| Sage essential oil | | | | | | | | 2,000 |
| Eucalyptus oil | | 1,000 | | 2,600 | | | | 1,000 |
| MCT oil (Miglyol 810) | 87,500 | 7,500 | 10,200 | 10,000 | 13,000 | 13,500 | 13,500 | 9,000 |
| DL-Tocopherol acetate | 2,000 | 2,500 | 2,500 | 2,750 | 3250 | 4250 | 4000 | 4750 |
| Lecithin | 1,000 | | 5,000 | 5,000 | 2500 | 3000 | 2500 | 3000 |
| Tocopherol acid succinate | | | | | 25 | 30 | 25 | 30 |
| Antioxidant mixture | | 60 | 60 | 60 | 25 | 30 | 25 | 30 |
| Total weight of the oil phase | 99,500 | 21,060 | 26,960 | 33,210 | 32,600 | 36,760 | 36,090 | 45,310 |
| Carbopol Ultrez ® 21 | | | | | 150 | 350 | 350 | 400 |
| Triethanolamine | | | | | 100 | 250 | 250 | 280 |
| Glycerin | | 2.6 | 2.4 | 2.4 | 2.4 | 2.4 | 2.4 | 2.4 |
| Antibacterial preservative | 500 | 500 | 500 | 500 | 500 | 500 | 500 | 500 |
| Water | 0 | to | to | to | to | to | to | to |
| | | 100 g | 100 g | 100 g | 100 g | 100 g | 100 g | 100 g |
| Preparation type | Ointment | Cream | Spray | Spray | Lotion | Cream- | Cream- | Cream- |
| | | | | | | gel | gel | gel |
| Joint pain relief | + − | + | + + | + + + | + + | + + + | + + + | + + + + |

### Example 6: Oral CBD-EM Formulations

As it was shown in previous embodiments, the eutectic mixtures with CBD prove significantly higher solubility in lipid vehicle and can be incorporated into oral dosage forms in higher concentrations and amounts.

TABLE 7
Oil based CBD formulations containing CBD EM for oral application

| | Ex. 37 | | Ex. 38 | | Ex. 39 | | Ex. 39 | | Ex. 40 | | Ex. 41 | |
|---|---|---|---|---|---|---|---|---|---|---|---|---|
| | mg | % | mg | % | mg | % | mg | % | mg | % | mg | % |
| Cannabidiol crystalline | 2,400 | 40.6 | 20,000 | 31.9 | 2,000 | 33.3 | 2,500 | 52.0 | 2,500 | 45.4 | 1,000 | 23.8 |
| (99.87%) | | | | | | | | | | | | |
| Maisine CC | | | 10,000 | 16.0 | 2,000 | 33.3 | | | | | | |
| Miglyol 810 (MCT) | | | 12,000 | 19.2 | | | 1,250 | 26.0 | 1,200 | 21.8 | 1,200 | 23.0 |
| L-Menthol | 1,000 | 16.9 | | | 400 | 6.7 | | | | | | |
| Peppermint oil FCC | | | | | | | 750 | 15.6 | 200 | 3.6 | 400 | 7.7 |
| Orange oil FCC | | | 2,000 | 3.2 | | | | | | | | |
| Lecithin | | | 2,000 | 3.2 | 200 | 3 3 | 200 | 4.2 | 200 | 3.6 | 200 | 3.8 |
| Soybean oil | 1,000 | 16.9 | | | 800 | 13.3 | | | 800 | 14.5 | | |
| Sucralose | | | 60 | 0.1 | | | | | | | | |
| Cocoa butter | | | | | | | | | | | 1,000 | 19.2 |
| Ethanol | | | 4,000 | 6.4 | | | | | | | | |
| Benzyl alcohol | | | 8,000 | 12.8 | | | | | | | | |
| Tocopherol acetate | 1,400 | 23.7 | 4,000 | 6.4 | 500 | 8.3 | | | 500 | 9.1 | 300 | 5.8 |
| d-alpha Tocopherol | 100 | 1.7 | 500 | 0.8 | 100 | 1.7 | 100 | 2.1 | 100 | 1.8 | 100 | 1.9 |
| Butylated hydroxytoluene | 5 | 0.1 | 50 | 0.1 | 5 | 0.1 | 5 | 0.1 | 5 | 0.1 | 5 | 0.1 |
| Ascorbyl palmitate | 5 | 0.1 | 50 | 0.1 | 5 | 0.1 | 5 | 0.1 | 5 | 0.1 | 5 | 0.1 |
| Total weight, mg | 5910 | 100.0 | 62,660 | 100.0 | 6010 | 100.0 | 4810 | 100.0 | 5510 | 100.0 | 4210 | 100.0 |

In one embodiment, the formulations were prepared by combining of initially prepared EM and the rest components of the composition at the temperatures 40-65° C.; in another embodiment CBD can be solubilized in a separately prepared mixture of all components.

In another embodiment, CBD-EM combination can be incorporated into different types of colloidal systems (e.g., pro-liposomes and liposomal suspensions).

TABLE 8
Liposomal formulations containing CBD EM

| | Ex. 42 | | Ex. 43 | | Ex. 44 | |
|---|---|---|---|---|---|---|
| Pro-liposomal composition | mg | % | mg | % | mg | % |
| Cannabidiol crystalline (99.87%) | 100 | 16.7 | 250 | 15.6 | 250 | 12.2 |
| Cetostearyl alcohol | | | 250 | 15.6 | 250 | 12.2 |
| Lecithin | 448 | 74.7 | 995 | 62.2 | 500 | 24.3 |
| Hydrogenated lecithin | | | | | 500 | 24.3 |
| Ethyl oleate | | | | | 200 | 9.7 |
| Cholesterol | 50 | 8.3 | 100 | 6.3 | 100 | 4.9 |
| Sodium deoxycholate | | | | | 250 | 12.2 |
| Antioxidant mixture | 2 | 0.33 | 5 | 0.31 | 5 | 0.24 |
| Pro-liposomal composition, total weight | 600 | 100% | 1600 | 100% | 2055 | 100% |

All components were dissolved in a hot (65° C.) ethyl alcohol (95% v/v), Obtained solution was slowly added to a warm water while intense mixing, sonicated for 30 minutes and passed through PTFE membrane syringe filter (0.45 and 0.2 mem) 5-8 times at 60° C. Prepared liposomal suspension can be freeze-dried with 10% mannitol solution in water.

The embodiments described herein are intended to be merely exemplary. Persons of ordinary skill in the art will understand that variations and modifications may be made without departing from the scope of the invention encompassed by the claims below.

1. A eutectic mixture comprising cannabidiol and at least one physiologically compatible hydroxyl containing organic compound, selected from the group consisting of aliphatic alcohols C8-C22, cyclic alcohols, aromatic alcohols, glycerides, polyethoxylated glycerides, polyethylene glycols, polyethylene glycol esters, phenols, tocopherols, phospholipids, and combinations thereof;
wherein the eutectic mixture liquefies at temperatures below 55° C.;
wherein the eutectic mixture comprises from about 20% to about 80% of cannabidiol by weight;
wherein the cannabidiol in the eutectic mixture remains in dissolved state during the extended storage at the temperatures equal or higher than melting point of the corresponding eutectic mixture;
wherein the cannabidiol containing eutectic mixture is essentially soluble in polar lipids, glycerides, non-ionic surfactants, alcohols, phospholipids and combinations thereof; and
wherein the eutectic mixture constitutes from about 5% to about 90% of a dosage form by weight.

2. The eutectic mixture of claim 1, wherein a molar ratio between the cannabidiol and the organic compound is between about 10:1 to about 1:20.

3. The eutectic mixture of claim 1, comprising cannabidiol and cetyl alcohol

4. The eutectic mixture of claim 1, comprising cannabidiol and behenyl alcohol

5. The eutectic mixture of claim 1, comprising cannabidiol and 5-methyl-2-(isopropyl)cyclohexan-1-ol.

6. The eutectic mixture of claim 1, comprising cannabidiol and methyl 2-hydroxybenzoate.

7. The eutectic mixture of claim 1, comprising cannabidiol and glyceryl monolinoleate.

8. The eutectic mixture of claim 1, comprising cannabidiol and glycerine monostearate.

9. The eutectic mixture of claim 1, comprising cannabidiol and glyceryl monocaprylate.

10. The eutectic mixture of claim 1, comprising cannabidiol and polyethylene glycol with molecular mass from about 200 to about 8000 dalton.

11. The eutectic mixture of claim 1, comprising cannabidiol and polyethoxylated fatty acid.

12. The eutectic mixture of claim 1, comprising cannabidiol and tocopherol or a tocopherol derivative.

13. The eutectic mixture of claim 1, comprising cannabidiol and polyethoxylated glyceride.

14. The eutectic mixture of claim 1, comprising cannabidiol and tocopheryl acid succinate polyethylene glycol ester (TPGS).

15. The eutectic mixture of claim 1, comprising cannabidiol and polyethoxylated sorbitan fatty acid ester (polysorbate).

16. The eutectic mixture of claim 1, comprising cannabidiol and cocoa butter.

17. The eutectic mixture of claim 1, comprising cannabidiol and 12-hydroxystearic acid.

18. The eutectic mixture of claim 1, comprising cannabidiol and lauroyl polyoxyl-32 glycerides.

19. The eutectic mixture of claim 1, comprising cannabidiol and caprylcaproyl polyoxyl (8) glycerides.

20. The eutectic mixture of claim 1, comprising cannabidiol and hydrogenated lecithin.

21. The eutectic mixture of claim 1, comprising cannabidiol, cocoa butter and menthol.

22. The eutectic mixture of claim 1, comprising cannabidiol, menthol and methyl 2-hydroxybenzoate.

23. The eutectic mixture of claim 1, comprising cannabidiol, glyceryl monolinoleate and polyoxyl glyceride.

24. The eutectic mixture of claim 1, comprising cannabidiol, glyceryl monolinoleate and polysorbate.

25. A pharmaceutical composition comprising the eutectic mixture of claim 1, wherein the eutectic mixture is incorporated in a pharmaceutical composition comprising lipids, phospholipids, non-ionic surfactants, antioxidants and other excipients or combination thereof; and wherein the cannabidiol is completely dissolved in the composition.

26. The pharmaceutical composition of claim 25, wherein the composition is a liquid solution of CBD in an oily based vehicle.

27. A method for increasing cannabidiol solubility for use in pharmaceutical formulations with high cannabidiol loading comprising a eutectic mixture comprising cannabidiol and at least one physiologically compatible hydroxyl containing organic compound, selected from the group consisting of aliphatic alcohols C8-C22, cyclic alcohols, aromatic alcohols, glycerides, polyethoxylated glycerides, polyethylene glycols, polyethylene glycol esters, phenols, tocopherols, phospholipids, and combinations thereof;
wherein the eutectic mixture liquefies at temperatures below 55° C.;
wherein the eutectic mixture comprises from about 20% to about 80% of cannabidiol by weight;
wherein the cannabidiol in the eutectic mixture remains in dissolved state during the extended storage at the temperatures equal or higher than melting point of the corresponding eutectic mixture;
wherein the cannabidiol containing eutectic mixture is essentially soluble in polar lipids, glycerides, non-ionic surfactants, alcohols, phospholipids and combinations thereof; and
wherein the eutectic mixture constitutes from about 5% to about 90% of a dosage form by weight.
